(19) United States
(12) Patent Application Publication
(10) Pub. No.: US 2019/0117511 A1
(43) Pub. Date: Apr. 25, 2019

(54) Secure Medication Storage System and Method of Securely Storing Medication in Healthcare Facility

(71) Applicant: Frantrice McMillan-Riley, Chicago, IL (US)

(72) Inventor: Frantrice McMillan-Riley, Chicago, IL (US)

(21) Appl. No.: 16/166,933

(22) Filed: Oct. 22, 2018

Related U.S. Application Data
(60) Provisional application No. 62/574,786, filed on Oct. 20, 2017.

(51) Int. Cl. A61J 1/14; G07C 9/00; G16H 20/13; A61J 7/00

(52) U.S. Cl. CPC A61J 1/1437; G07C 9/0069; A61J 2205/60; G16H 20/13; A61J 7/0069; G07C 9/00912

(57) ABSTRACT

A device for securely storing medication within a healthcare facility is provided. The device may include an enclosure sized and adapted to be mounted in a patient room of the healthcare facility, a door movable attached to the enclosure and having open and closed configurations, the door and enclosure defining a storage space for medication when in the closed configuration, at least one shelf provided in the enclosure, at least one tray attached to an inside surface of the door, a handle on the outside of the door, and a lock adapted to secure the door to the enclosure when in a locked configuration and allow access to the storage space when in an unlocked configuration.

### CROSS-REFERENCE TO RELATED APPLICATION

This application is a non-provisional US patent application claiming priority under 35 USC 119(e) to U.S. Provisional Patent Application Ser. No. 62/574,786 filed on Oct. 20, 2017.

### TECHNICAL FIELD

The present disclosure generally relates to healthcare facilities and, more particularly, relates to systems and methods for storing medication within a healthcare facility.

### BACKGROUND

The storage of medication with a healthcare facility in a secure, reliable, and sanitary fashion is a significant issue facing the medical community. For example, using a hospital as one example of a healthcare facility, the main supply of medication for the hospital is typically provided in a centralized pharmacy. When a doctor or other healthcare provider prescribes a given medication to a patient, a nurse or other healthcare provider physically goes to the pharmacy to gather the necessary medication dispensed. A pharmacist or other healthcare provider dispenses the medication to the nurse, who then transports the medication to the patient room housing the patient.

If timely, the medication is immediately given to the patient by the nurse, or even another healthcare provider, upon arrival into the patient room. Often times however, the medication is not yet called for given the dosage required, or the prescribed timing between doses. As a result, the medication may simply wait in the patient room. Other times, a medical emergency may occur, either involving the patient in question or another patient within the healthcare facility. With such an occurrence, the medication is again often simply left waiting in the patient room in an unsecure and unsanitary fashion.

From the foregoing it can therefore be seen that given the current construct of healthcare facilities, which are not limited to hospitals, but can also include medical centers, doctor's offices, nurse's offices, rehabilitation facilities, treatment centers, clinics and like, multiple situations can arise where prescribed medications can be left unattended. This causes a number of resulting difficulties. For example, the medication may be misplaced in the interim, taken by a visitor or someone other than the proper patient, or if left unattended by the healthcare provider, taken at an inappropriate time or not taken at all. Moreover, the potential for cross-contamination of such medications is huge. Single use medications with multiple doses may be transported from room to room allowing various patients to either contaminate the remainder. This may be done either by way of physical contact or by way of airborne transport while be carried back to the central pharmacy.

In addition, the dispensed medications are often currently housed in plastic containers, and given that such enclosures are porous, the potential for contamination is also increased in this fashion as well. To the extent the medication is transported back to the central pharmacy, it is likely only stored in a drawer or cassette labeled for the given patient. These drawers are used by nurses maintaining the pharmacy and not cleaned between patient assignments to that drawer. As medications are returned to the drawer, there is no known protocol for sanitizing the medication containers or drawers in which they are stored. This transport process creates a high potential for cross-contamination as medication containers are transported from the patient rooms and returned to the designated medication drawer in the pharmacy, and/or transported back to the patient room and placed on a potentially compromised surface therein.

Another problem associated with medications within healthcare facilities is that such medications are often narcotics or otherwise highly addictive. If left unattended, the opportunity for theft or unprescribed ingestion increases. Moreover, government regulations and Joint Commission Compliance often require such medications to be securely stored and accounted for at all times.

In view of the foregoing disadvantages associated with conventional storage systems and methods, a need exists for better systems and methods for securely storing medication within healthcare facilities.

### SUMMARY OF THE DISCLOSURE

In one aspect of the present disclosure, a system for securely storing medication in a healthcare facility is provided. The system may include a healthcare facility having a plurality of patient rooms, each patient room having at least one wall, a plurality of medication storage devices with one of the plurality of medication storage devices mounted in each at least one wall, each medication storage device having an enclosure with a movable door mounted thereto and a lock adapted to hold the door in a locked configuration and release the door in an unlocked configuration, a centralized pharmacy located within the healthcare facility, and a supply of medication stored in the centralized pharmacy and the plurality of medication storage devices.

In another aspect of the present disclosure, a device for securely storing medication within a healthcare facility is provided. The device may include an enclosure sized and adapted to be mounted in a patient room of the healthcare facility, a door movable attached to the enclosure and having open and closed configurations, the door and enclosure defining a storage space for medication when in the closed configuration, at least one shelf provided in the enclosure, at least one tray attached to an inside surface of the door, a handle on the outside of the door, and a lock adapted to secure the door to the enclosure when in a locked configuration and allow access to the storage space when in an unlocked configuration.

In yet another aspect of the present disclosure, a method of storing medication within a healthcare facility having a plurality of patient rooms and a centralized pharmacy is provided. The method may include mounting a medication storage device in a wall of each of the plurality of patient rooms, storing a supply of medication in the centralized pharmacy, dispensing the supply of medication from the centralized pharmacy to a healthcare professional, having the healthcare professional transport the supply of medication to one of the plurality of patient rooms, unlocking the medication storage device in the patient room, depositing the supply of medication into the medication storage device, locking the medication storage device until the medication is needed by a patient in the patient room.

These and other aspects and features of the present disclosure will be better understood upon reading the following detailed description with read in conjunction with the accompanying drawings.

### BRIEF DESCRIPTION OF THE DRAWINGS

FIG. 1 is a block diagram of a system for storing medication in a healthcare facility according to the present disclosure;

FIG. 2 is perspective view of a medication storage device according to the present disclosure, and depicted in a locked configuration;

FIG. 3 is a perspective view of the medication storage device of FIG. 2 but depicted in an unlocked configuration;

FIG. 4 is front view of the medication storage device of FIG. 2;

FIG. 5 is a plan view of an inside of a door of the medication storage device of FIG. 2;

FIG. 6 is a front view of an inside of an enclosure forming part of the medication storage device of FIG. 2;

FIG. 7 is a flowchart depicting a sample sequence of steps that may be practiced in accordance with the teachings of the present disclosure.

Although the following sets forth a detailed description of numerous different embodiments, it should be understood that the legal scope of protection is defined by the words of the claims set forth at the end of this patent. The detailed description is to be construed as exemplary only and does not describe every possible embodiment since describing every possible embodiment would be impractical, if not impossible. Numerous alternative embodiments could be implemented, using either current technology or technology developed after the filing date of this patent, which would still fall within the scope of the claims defining the scope of protection.

It should also be understood that, unless a term is expressly defined herein, there is no intent to limit the meaning of that term, either expressly or by implication, beyond its plain or ordinary meaning, and such term should not be interpreted to be limited in scope based on any statement made in any section of this patent other than the language of the claims. To the extent that any term recited in the claims at the end of this patent is referred to herein in a manner consistent with a single meaning, that is done for sake of clarity only so as to not confuse the reader, and it is not intended that such claim term be limited, by implication or otherwise, to that single meaning.

### DETAILED DESCRIPTION

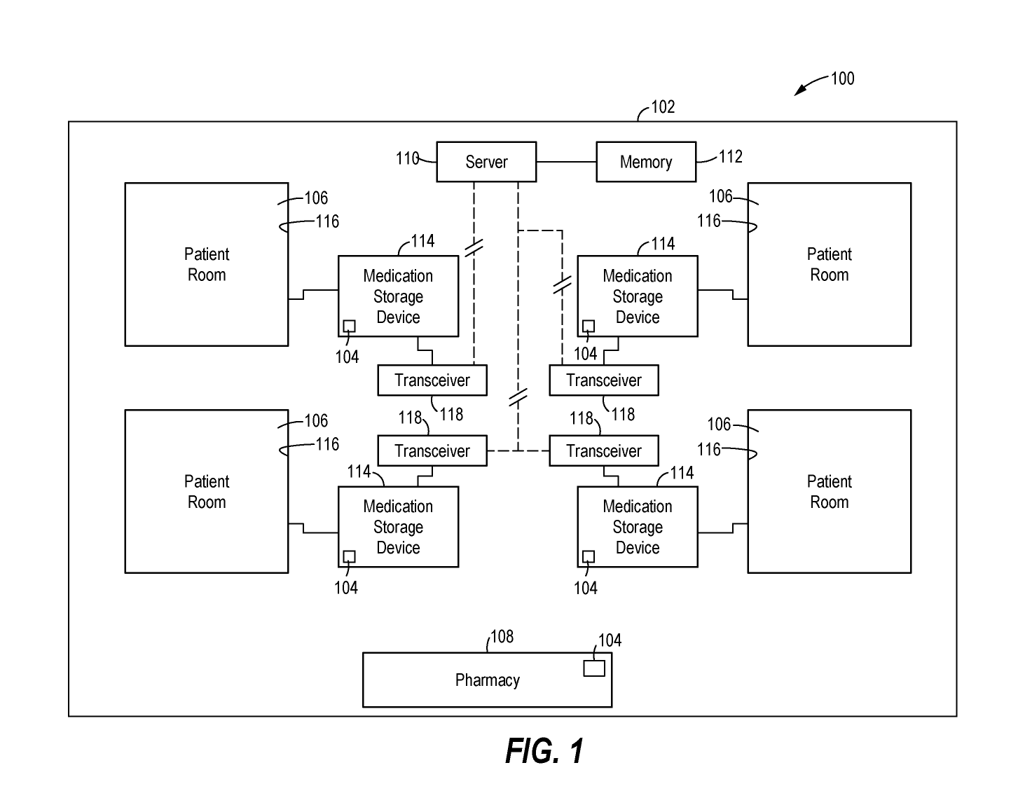

Referring now to FIG. 1, a system for securely storing medication is generally referred to by reference numeral 100. While the system 100 is depicted as securely storing medication with a healthcare facility 102, the system 100 can also be used within homes and commercial establishments as well. Within the context of healthcare facility, such facilities include but are not limited to hospitals, medical centers, doctor's offices, nurse's offices, clinics, rehabilitation centers, treatment centers and the like. Any location needing secure and sanitary storage of medication can benefit from system 100.

As shown in the drawings, the system 100 may be provided within the healthcare facility 102 for securely storing medication 104. The healthcare facility may include a plurality of patient rooms 106. While four (4) such patient rooms 106 are depicted, it is to be understood that this is only exemplary and that more or less rooms may be provided.

The healthcare facility 102 may also include a pharmacy 108 which may be centrally located or otherwise accessible by healthcare providers within the hospital. The pharmacy includes a supply of many different types of medication 104 that may be dispensed by a pharmacist or other healthcare provider upon receipt of instructions to do so by a physician, nurses or other healthcare provider (not shown). A computer server 110 or servers 110 with associated memory 112 may also be provided as part of the system 100 as will be described in greater detail herein.

Within each patient room 106, at least one secure medication device 114 is provided. In one embodiment, each room 106 includes at least one wall 116 in which the secure medication device 114 is mounted. It may be recessed within the wall to be flush therewith, or be otherwise mounted therein. In another embodiment, the secure medication device may be bolted or otherwise fastened to a fixed surface or counter within the room 106. Each medication storage device 114 may have a transceiver 118 operatively associated therewith as will be described in further detail herein.

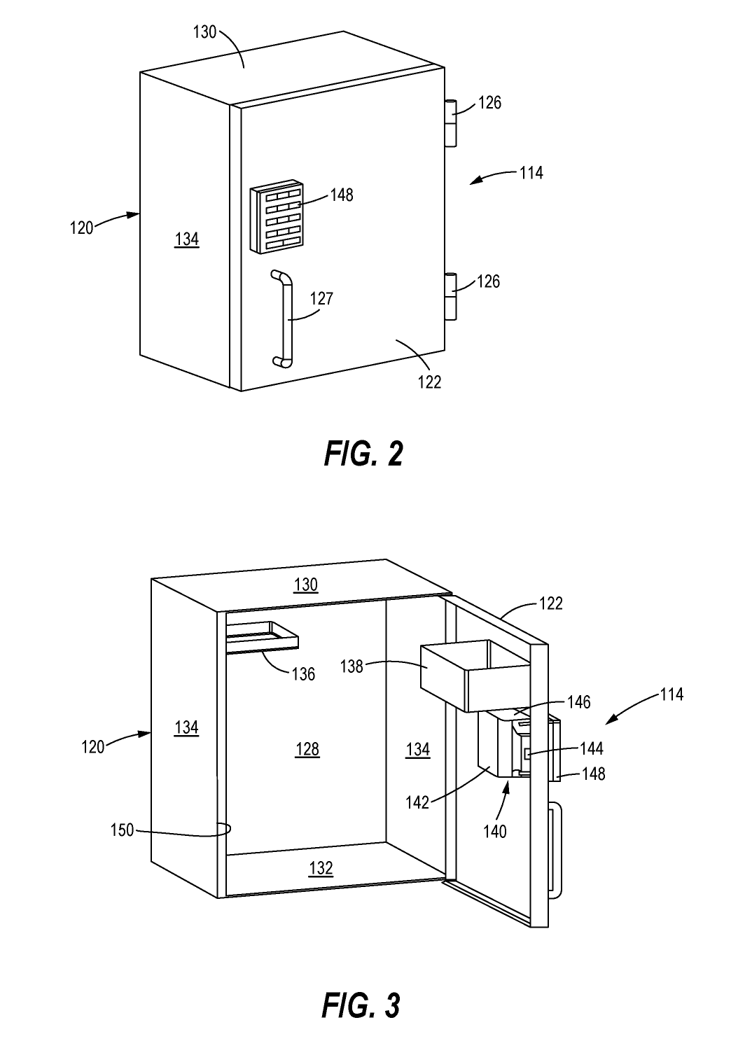

Turning now to FIG. 2, one embodiment of the secure medication device 114 is shown in detail. As illustrated therein, the secure medication device 114 may be substantially box-like in three dimensional shape, with a rectangular cross-section. Of course, this is only exemplary and any number of other geometrical shapes including, but not limited to, square, parallelepiped, spherical, triangular, pyramidal, quadrilateral, multilateral, or amorphous are possible.

The secure medication device 114 includes an enclosure 120, with a door 122 movable associated therewith. Together, the enclosure 120 and door 122 define a storage space 124 for receipt and storage of the medication 104. While the door 122 is shown as being hingedly attached to the enclosure 120 with hinges 126, it is to be understood that this too is only exemplary and that may other ways of movably associating the door 122 with the enclosure 120 are possible. For example, the door 122 may slide relative to the enclosure, the door 122 may be completely removable from the enclosure and held only with friction, clasps or the like, the door may roll up relative to the enclosure, may accordion fold, may have clamshell halves, or be provided in myriad other ways to allow access to the storage space 124.

It is also important to note that the enclosure 120 is seamless. More specifically, the enclosure 120 includes a back panel 128, a top panel 130, a bottom panel 132, and two side panels 134, all of which may be made of a unitary piece of material. By “seamless”, applicants mean that the transitions between the back panel 128 and the top, bottom and side panels do not include any opening, crevices or significant surface deformations. In doing so, the enclosure can be easily cleaned and kept antiseptic. Moreover, to facility the sterility of the enclosure 120, the enclosure may be manufacture from metal, such as stainless steel. Other metals and materials are certainly possible for similar effect.

As shown best in FIGS. 3, 5 and 6, the enclosure 120 and door 122 may also be provided with one or more storage shelves 136 and trays 138. While only one shelf 136 and one tray 138 are depicted it is to be understood that many of each may be provided. Moreover, the shelves 136 and trays 138 may be provided at other positions with the storage device 114. Of course, other structures may also be used to facilitate storage within the device 114 such as but not limited to drawers, hooks, folding ledges, and the like.

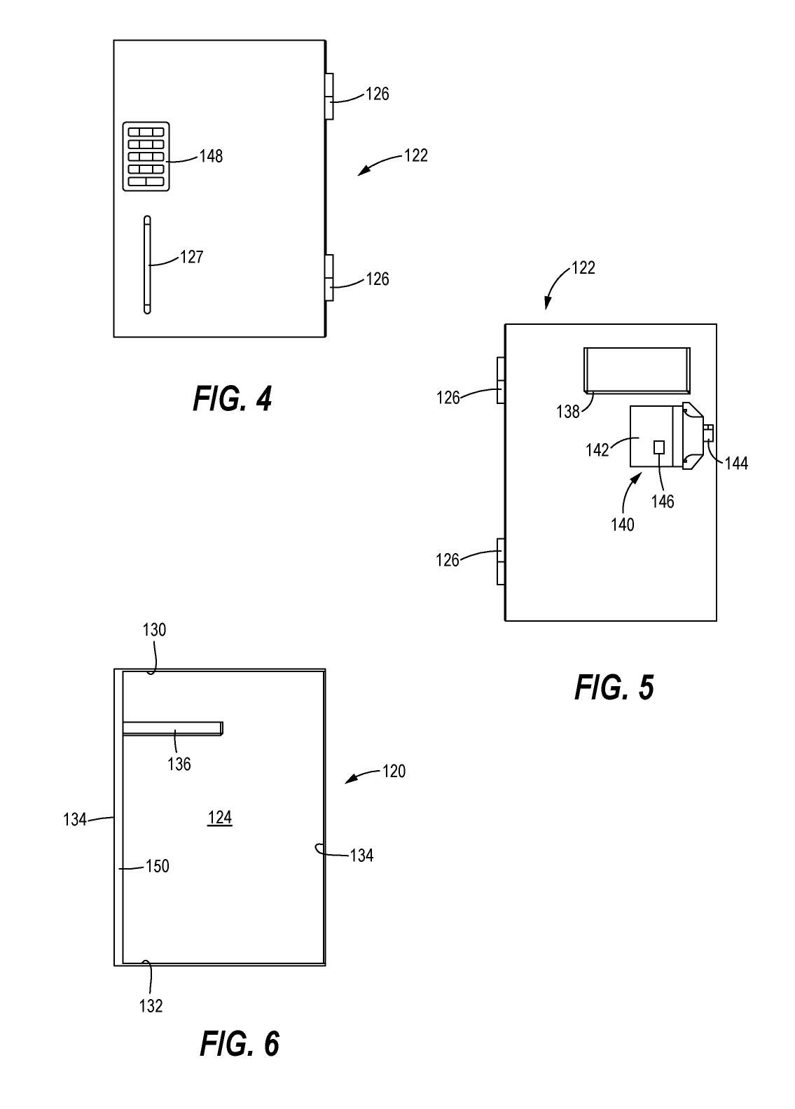

Referring now to FIG. 4, it can be seen that the medication storage device 114 also includes a lock 140. One embodiment of the lock 140 is depicted but may other locks configurations are possible for securing the door 122 to the enclosure 120 and only enabling access when desired. With respect to the depicted embodiment, however, the lock 140 may include a housing 142 relative to which a locking bar 144 is adapted to reciprocatingly slide. Moreover, the lock 140 may include a processor 146 and keypad 148. Upon entry of the appropriate passcode into the keypad 148, the processor 146 may cause the locking bar 144 to slide between open and closed configurations. The enclosure may include an inwardly directed flange 150 to cooperate with the locking bar 144 and prevent movement of the door 122 when the bar 144 is in the locked configuration.

While the depicted embodiment includes an electronic keypad 148 it is to be understood that many other forms of input/output devices are possible including, but not limited to biometric readers, fingerprint readers, retinal scanners and face recognition software. In addition, mechanical or electric keys could be used, or radio frequency fobs with associated RFID tags could be used to lock and unlock the device 114 when in proximity thereto.

Finally, it will be noted both from FIG. 1 and FIG. 5, that the lock medical storage device 114 and lock 140 specifically, may include the electronic transceiver 118. The electronic transceiver 118 may be in wireless communication with the healthcare facility server 110 so as to remotely monitor and track the opening and closing of each storage device 114. Moreover, if each healthcare provider wears an identification badge including an RFID tag or the like, the transceiver 118 can monitor and track who is opening the devices 114, and when, as well. Similarly, if the shelves or trays are equipped with weight or movement sensors, this data can be received by the servers to enable tracking of each dosage dispensed as well.

Other variations and modifications of devices, systems and methods disclosed herein will be apparent to those of ordinary skill in the art.

### INDUSTRIAL APPLICABILITY

In operation, the present disclosure can find applicability in a variety of different industries. For example, the devices, systems and methods disclosed herein may be employed to effectively and securely store medication in healthcare facilities including, but not limited to, hospitals, medical centers, nursing homes, doctor's offices, school nurse's offices, infirmaries, treatment centers, rehabilitation facilities, clinics, and the like.

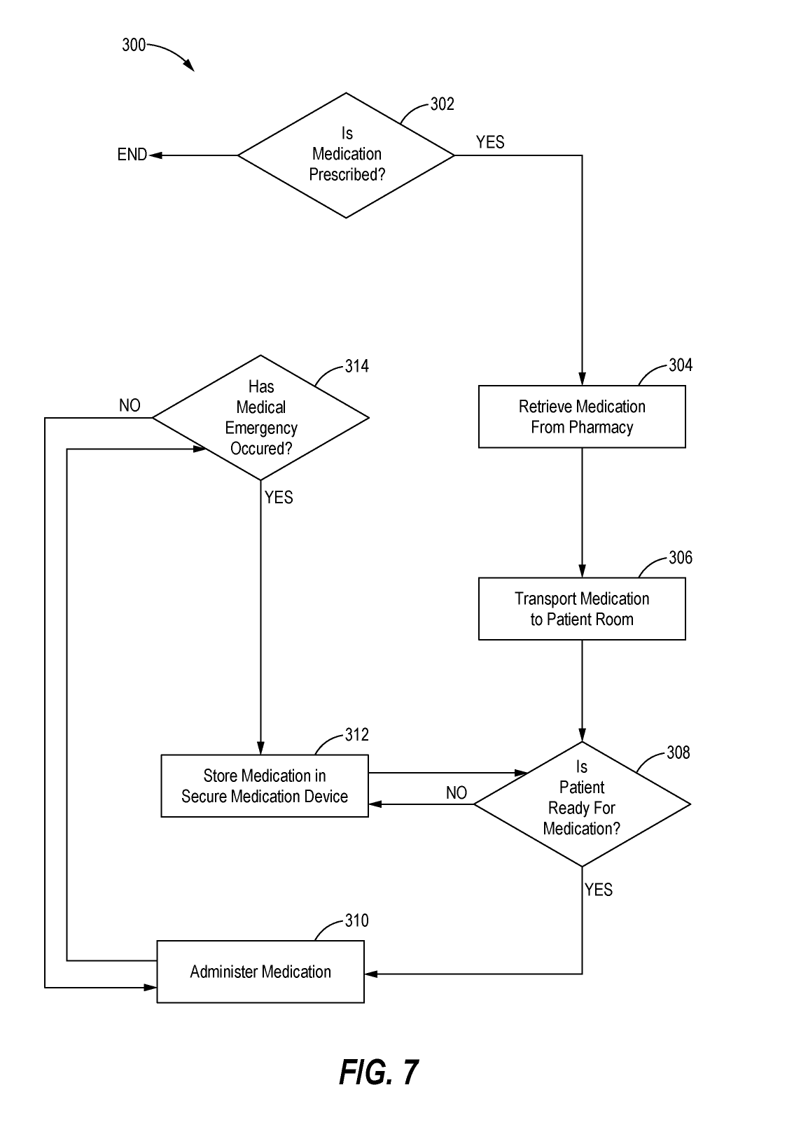

One exemplary method 300 for securely storing medication in a healthcare facility is diagrammatically provided in the flow chart depicted in FIG. 7. As shown therein, the method may begin with a block 302 inquiring if a medication has been prescribed by a healthcare provider. If yes, another healthcare provider is dispatched to retrieve the necessary medication or medications 104 from the facility pharmacy as depicted by block 304. If not, the method ends. Once retrieved from the pharmacy, the medication is transported by a healthcare provider to the patient room as shown by block 106.

Once within the patient room, the method inquires whether the patient is immediately ready for the medication at block 306. If yes, the medication is administered to the patient as shown by block 308. If the patient is not ready for medication, based on the dosage and timing prescribed by the healthcare provider or the activities within the patient room, the medication may be stored in the secure medication device as shown by block 310. As will be apparent based on the foregoing, this may require the healthcare provider to enter a code into the keypad, effect a biometric reading, be close enough for the RFID tag to work or the like. Once so opened, the medication can be securely stored until such time as it is needed.

For example, at a block 312, an inquiry is made after storage as to whether the patient is now ready for the medication. This inquiry can be repeatedly made until such time that the answer is in the affirmative whereupon the storage device can be reopened and the medication can be administered.

It should also be noted that ever after the medication storage device is opened and the medication is about to me administered or is actually being administered, given the nature of healthcare facilities, a medical emergency may arise as show by block 314. In such an event, the medication can again be secured stored in the medication storage device until safe or appropriate to readminister.

By providing such a system, method and device, the secure and sanitary storage and dispensing of medication within a healthcare facility can be effected. This will reduce the spread of infection and bacteria, maintain the integrity and safety of medication supplies, and ensure compliance with governmental and other regulatory bodies. Moreover, such a system will mitigate the ease with which narcotics and other addictive drugs can be taken, but in healthcare facilities and with home usage alike.

From the foregoing, it will be appreciated that while only certain embodiments have been set forth for the purposes of illustration, alternatives and modifications will be apparent from the above description to those skilled in the art. These and other alternatives are considered equivalents and within the spirit and scope of this disclosure and the appended claims.

What is claimed is:

1. A device for storing medication in a healthcare facility, comprising:
an enclosure sized and adapted to be mounted in a patient room of the healthcare facility;
a door movable attached to the enclosure and having open and closed configurations, the door and enclosure defining a storage space for medication when in the closed configuration;
at least one shelf provided in the enclosure;
at least one tray attached to an inside surface of the door;
a handle on the outside of the door; and
a lock adapted to secure the door to the enclosure when in a locked configuration and allow access to the storage space when in an unlocked configuration.

2. The device of claim 1 wherein the enclosure, door, shelf, tray and handle are made of metal.

3. The device of claim 2, wherein the metal is stainless steel.

4. The device of claim 3, wherein the enclosure is seamless.

5. The device of claim 1, wherein the lock includes a keypad enabling a passcode to be entered to unlock the device.

6. The device of claim 1, wherein the device is adapted to mounted into a wall of the patient room.

7. The device of claim 1, wherein the device is adapted to mounted onto a counter of the patient room.

8. A system for securely storing medication in a healthcare facility, comprising:
a healthcare facility having a plurality of patient rooms, each patient room having at least one wall;
a plurality of medication storage devices with one of the plurality of medication storage devices mounted in each at least one wall, each medication storage device having an enclosure with a movable door mounted thereto and a lock adapted to hold the door in a locked configuration and release the door in an unlocked configuration;
a centralized pharmacy located within the healthcare facility; and
a supply of medication stored in the centralized pharmacy and the plurality of medication storage devices.

9. The system of claim 8, wherein each medication storage device further includes at least one shelf within the enclosure, at least one tray mounted to inside of the door of the device, and a handle mounted to an outside of the door.

10. The system off claim 8, wherein the enclosure, door, shelf, tray and handle are made of metal.

11. The system of claim 10, wherein the metal is stainless steel.

12. The system of claim 11, wherein the each enclosure is seamless.

13. The system of claim 8, wherein the lock includes a keypad enabling a passcode to be entered to unlock the device.

14. A method of storing medication within a healthcare facility having a plurality of patient rooms and a centralized pharmacy, comprising:
mounting a medication storage device in a wall of each of the plurality of patient rooms;
storing a supply of medication in the centralized pharmacy;
dispensing the supply of medication from the centralized pharmacy to a healthcare professional;
having the healthcare professional transport the supply of medication to one of the plurality of patient rooms;
unlocking the medication storage device in the patient room;
depositing the supply of medication into the medication storage device;
locking the medication storage device until the medication is needed by a patient in the patient room.

15. The method of claim 12, further including unlocking the medication storage device when the medication is needed, using the medication, and restoring any remainder of the medication in the medication storage device, and relocking the medication storage device.

16. The method of claim 13, further including storing medication in the medication storage device when a patient emergency arises.

17. The method of claim 13, wherein each medication storage device further includes at least one shelf within the enclosure, at least one tray mounted to inside of the door of the device, and a handle mounted to an outside of the door.

18. The method of claim 17, wherein the enclosure, door, shelf, tray and handle are made of metal.

19. The method of claim 18, wherein the metal is stainless steel.

20. The method of claim 19, wherein the each enclosure is seamless.
